(19) United States
(12) Patent Application Publication
(10) Pub. No.: US 2025/0360088 A1
(43) Pub. Date: Nov. 27, 2025

(54) CHARGE MODIFIED CHITOSAN CROSS-LINKED ENCAPSULATE

(71) Applicant: Encapsys, LLC, Appleton, WI (US)

(72) Inventors: Linsheng Feng, Menasha, WI (US); Robert Stanley Bobnock, Menasha, WI (US); Travis Ian Bardsley, Appleton, WI (US); Johan Smets; Cedric Marc Tahon; Mattia Collu

(73) Assignee: Encapsys, LLC, Appleton, WI (US)

(21) Appl. No.: 18/871,645

(22) PCT Filed: Nov. 29, 2023
(86) PCT No.: PCT/US2023/081490

§ 371 Date: Dec. 4, 2024

Related U.S. Application Data
(60) Provisional application No. 63/429,227, filed on Dec. 1, 2022.

(51) Int. Cl. A61K 9/50

(52) U.S. Cl. CPC A61K 9/5089; A61K 9/5036

(57) ABSTRACT

An improved polyurea and chitosan core-shell delivery particles encapsulating a benefit agent is described. Chitosan is pre-modified with a modifying compound which is cationic, anionic, or nonionic. Alternatively the modification is accomplished in situ. The modifying compound is selected from the group consisting of an epoxide, aldehyde, or an α,β-unsaturated compound, and is reactive with free amine moieties of chitosan, covalently bonding through CN bonds with the amine moieties of the chitosan. The modifying compound can contain acidic, hydroxyl, and quaternary ammonium groups. The reaction product of the chitosan, the modifying compound along with an electrophile, preferably a polyisocyanate yields a microcapsule with charge, improved release characteristics, improved compatibility or enhanced degradation characteristics, such as in OECD test method 301B.

### CROSS REFERENCE TO RELATED APPLICATIONS

### Field of the Invention

This invention relates to manufacturing processes for delivery particles and delivery particles produced by such processes.

### Description of the Related Art

Various processes for encapsulation, specifically microencapsulation, and exemplary methods and materials are set forth in Schwantes (U.S. Pat. No. 6,592,990), Nagai et al. (U.S. Pat. No. 4,708,924), Baker et al. (U.S. Pat. No. 4,166,152), Wojciak_(U.S. Pat. No. 4,093,556), Matsukawa et al. (U.S. Pat. No. 3,965,033), Matsukawa (U.S. Pat. No. 3,660,304), Ozono (U.S. Pat. No. 4,588,639), Irgarashi et al. (U.S. Pat. No. 4,610,927), Brown et al. (U.S. Pat. No. 4,552,811), Scher (U.S. Pat. No. 4,285,720), Shioi et al. (U.S. Pat. No. 4,601,863), Kiritani et al. (U.S. Pat. No. 3,886,085), Jahns et al. (U.S. Pat. Nos. 5,596,051 and 5,292,835), Matson (U.S. Pat. No. 3,516,941), Chao (U.S. Pat. No. 6,375,872), Foris et al. (U.S. Pat. Nos. 4,001,140; 4,087,376; 4,089,802 and 4,100,103), Greene et al. (U.S. Pat. Nos. 2,800,458; 2,800,457 and 2,730,456), Clark (U.S. Pat. No. 6,531,156), Saeki et al. (U.S. Pat. Nos. 4,251,386 and 4,356,109), Hoshi et al. (U.S. Pat. No. 4,221,710), Hayford (U.S. Pat. No. 4,444,699), Hasler et al. (U.S. Pat. No. 5,105,823), Stevens (U.S. Pat. No. 4,197,346), Riecke (U.S. Pat. No. 4,622,267), Greiner et al. (U.S. Pat. No. 4,547,429), and Tice et al. (U.S. Pat. No. 5,407,609), among others and as taught by Herbig in the chapter entitled “Microencapsulation” in Kirk-Othmer Encyclopedia of Chemical Technology, V.16, pages 438-463.

### STATEMENT OF JOINT RESEARCH

Encapsys, LLC and The Procter & Gamble Company executed a Joint Research Agreement on or about Jul. 29, 2021 and this invention was made as a result of activities undertaken within the scope of that Joint Research Agreement between the parties that was in effect on or before the date of this invention.

Each patent described throughout this application is incorporated herein by reference to the extent each provides guidance regarding microencapsulation processes and materials.

Jabs et al., U.S. Pat. No. 4,847,152 teaches microcapsules with polyurea walls. The wall is the reaction product of an aromatic isocyanate with an isocyanate reactive group. The isocyanate reactive group can include di- and polyamines such as N-hydroxyethylethylenediamine, ethylene-1,2-diamine.

Hotz et al., U.S. Pat. Pub. 2013/0089590 teaches a fragrance microcapsule with a polyurea wall. The shell in the reaction product of at least two difunctional isocyanates and a difunctional amine.

EP 1693104 Maruyyama discloses microcapsules having a polyurethane or polyurea wall obtained from polycondensation of a polyfunctional isocyanate with a polyfunctional amine.

U.S. Pat. No. 9,816,059 describes a polyurea capsule, the capsule encapsulating an oil core, where the polyurea is a reaction product of a polyfunctional isocyanate and a polyfunctional amine. The polyfunctional amine can include hexamethylene diamine and other amines including chitosan. Chitosan is mentioned as a stabilizing agent, as a polyfunctional amine, as a coating, without any guidance or example how to work with this difficult to handle material.

Chitosan is a polysaccharide and can be a difficult material to utilize in microencapsulation processes. Chitosan is generally insoluble in water above pH 7, and below about pH 6.5 is cationic. Chitosan is soluble in low pH acidic solutions such as hydrochloric acid, lactic acid, propionic acid, succinic acid, acetic acid, citric acid, and phosphoric acid, forming a hard to handle viscous solution but generally insoluble in water above pH 7. At pH values below 4, the amino groups of chitosan promote electrostatic repulsion and the polymer swells.

The dissolved polysaccharide has positive charged —NH₃⁺ groups and adheres to anionic surfaces. Chitosan forms aggregates with polyanions and chelates heavy metals.

A need exists in the art for polyurea type delivery particles having improved properties in terms of better deposition efficiency, lower leakage measured as lower free oil, and having cationic charge at pH less than about 7. If chitosan can be adapted to be useful as a solubilized cross-linker, an improved polyurea wall material becomes possible.

The present invention overcomes the above deficiencies of the present art by teaching an improved polyurea delivery particle cross-linked with chitosan. The chitosan is hydrolyzed by treating with acid to enable the chitosan to be soluble even at pH above 5, enabling its use in microencapsulation processes such as interfacial encapsulation.

Although the art generally mentions chitosan as a possible component in forming wall material in microencapsulation, there is little teaching as how to practically utilize this difficult to handle material.

Chitosan is generally insoluble in water, alkali, and most organic solvents. Even under acidic low pH condition, solubility is generally less than 2 wt %. The composition is viscous, difficult to handle and requires considerable dilution. Chitosan concentrations less than 2 wt % make the material unsuitable for interfacial microencapsulation.

Chitosan is insoluble at higher pH and capsule formation under capsule forming conditions usually involves pH of 7 or 9 or even more alkaline conditions, presenting a situation where chitosan is an essentially insoluble viscous mass unsuitable for interfacial encapsulation.

A need exists for chitosan polyurea compositions at higher concentrations of chitosan which overcome the technical challenges of working with chitosan and provide a useful concentration greater than 2 wt % in the water phase to enable successful chitosan urea polymer shell formation.

Although chitosan is mentioned as a cross-linker to prepare polyurea capsules such as in Lei et al., 2013/0330292, Lei does not provide any description how to employ chitosan. Chitosan is only soluble at low pH and not soluble at higher pH levels. As pH is increased, chitosan precipitates out of solution. Also, due to its high molecular weight, chitosan is an exceedingly difficult material to use as a cross-linker.

Bulgarelli et al., WO 2019063515 attempts to overcome the shortcomings of Lei by adding chitosan in solid form. Bulgarelli teaches adding chitosan in the water phase of the emulsion. Unprotonated chitosan is added once a reaction temperature of 80° C. is reached. The claims state the chitosan is added in solid form however, Bulgarelli provides no teaching in an example of how to effect dissolution of the solid chitosan. Chitosan is known to precipitate at alkaline pH's or even pH's exceeding 5.

Polyurea delivery particles have been described for certain applications as advantageous for being free of formaldehyde. Mechanical properties of polyurea systems described to date have not had core retention properties needed in certain challenging applications such as detergents, cleaners, compositions with surfactants, modifiers or other materials tending to negatively influence capsule performance upon prolonged storage. A polyurea chitosan that successfully incorporates chitosan at higher concentrations than heretofore achievable, that is stable in various matrix materials, that can be modified to create an encapsulate with a tailored surface charge, or that exhibits lower leakage would be an advance in the art. Improved shelf stability, lower leakage and degradability of such resultant compositions would be beneficial.

Crosslinked chitosan capsules although having certain benefits such as based on biocompatible materials, suffer from certain drawbacks under certain conditions of use. For example, chitosan capsules have been found to show poor compatibility with certain matrices such as in laundry detergent matrices particularly liquid laundry detergents.

The present invention solves the problem of incompatibility of crosslinked chitosan capsule slurries in laundry matrices. The present invention teaches crosslinked chitosan capsules that are able to be modified to bear a surface charge.

Through modification of the surface charge of encapsulates to increase or decrease zeta potential, incompatibility with certain matrices can be overcome. Aggregation can be reduced or even eliminated, along with reduced leakage.

### Definitions

For ease of reference in this specification and in the claims, the term “monomer” or “monomers” as used herein with regard to the structural materials that form the wall polymer of the delivery particles is to be understood as monomers, but also is inclusive of oligomers and/or prepolymers formed of the specific monomers.

As used herein the term “water soluble material” means a material that has a solubility of at least 0.5% wt in water at 60° C.

As used herein the term “oil soluble” means a material that has a solubility of at least 0.1% wt in the core of interest at 50° C.

As used herein the term “oil dispersible” means a material that can be dispersed at least 0.1% wt in the core of interest at 50° C. without visible agglomerates.

As used herein, “delivery particles,” “particles,” “encapsulates,” “microcapsules,” and “capsules” are used interchangeably, unless indicated otherwise. As used herein, these terms typically refer to core/shell delivery particles. “Shell” and “wall” are also interchangeably used to refer to the shell of the core/shell delivery particles.

### SUMMARY OF THE INVENTION

The present invention teaches a capsule shell polymeric material which is comprised of an oil soluble crosslinker and a modified chitosan polymer. The oil soluble crosslinker can be selected from a bifunctional or multifunctional isocyanate, acrylate, methacrylate, or acid chloride. Chitosan has free amine moieties. A modified chitosan polymer according to the invention comprises a natural chitosan polymer combined with a modifying compound that can form C—N covalent bonds with the amine moieties of chitosan, particularly primary or secondary amines. The modifying compound can be selected from an epoxide compound, an aldehyde compound or an α,β-unsaturated compound. The epoxide, aldehyde compound or α,β-unsaturated compound can be anionic, cationic, or nonionic. The modifying compound can contain acidic, hydroxyl, or quaternary ammonium groups. The α,β-unsaturated compound can be selected from acrylates, alkyl acrylates, α,β-unsaturated esters, acrylic acid, acrylamides, vinyl ketones, vinyl sulfones, vinyl phosphonates, acrylonitrile derivatives or mixtures thereof. It was found that the modified chitosan polymer according to the invention is soluble at pH above 6.0, even above 8.0, even further above 10.0.

Advantageously, the surface charge of the crosslinked chitosan capsule can be modified and tailored before, during or after the formation of the capsule shell. This is accomplished by selection of the modifying compound and timing of the addition of the modifying compound. The addition can be to the emulsion or the water phase. In particular, enhanced surface charge in the capsule shell results when the modifying compound is selected to have cationic or anionic groups.

The invention teaches a process of forming a population of delivery particles, the delivery particles comprising a core and a shell surrounding the core, the core comprising a benefit agent and an oil phase, wherein the shell comprises a reaction product of at least one modified chitosan and at least one polyisocyanate.

The process of the invention comprises forming a water phase by dissolving chitosan and a modifying compound in an aqueous acidic medium at a pH of 6.5 or less and a temperature of at least 25° C. After the chitosan is modified with the modifying compound, the pH of the water phase, comprising the modified chitosan solution, can be adjusted to above 6.5, or even above 7, or even above 9 if needed.

The process steps also comprise forming an oil phase comprising combining together at least one benefit agent and at least one polyisocyanate, optionally with an added oil. An emulsion is formed by mixing under high shear agitation the oil phase into an excess of the water phase, thereby forming droplets of the oil phase and benefit agent dispersed in the water phase.

A water soluble or dispersible modifying compound is added to the emulsion or the water phase at room temperature or at elevated temperature. The modifying compound can be added during emulsification such as after milling or added thereafter at elevated temperature. The modifying compound contains cationic, anionic, or nonionic groups, typically selected from one or more acidic, or quaternary ammonium functional groups. In the process of the invention, the modifying compound, namely an epoxide, aldehyde or α,β-unsaturated compound is reacted with free amine moieties of chitosan.

Optionally the pH of the emulsion is adjusted to a pH of 4 or greater, or even to a pH of 6, or even 8 or even to 8-10 or higher alkalinity. The emulsion is heated to at least 40° C., for a time sufficient to form a shell at an interface of the droplets with the water phase, the shell surrounding the core. The delivery particles formed according to the process of the invention, particularly when the modifying compound has cationic or anionic groups, results in the shell of the delivery particles having a surface charge. In certain embodiments, such surface charged delivery particles have a zeta potential of 150 mV or less at pH 4.5.

The modifying compound useful in the process of the invention is selected from the group consisting of an epoxide, aldehyde, or an α,β-unsaturated compound containing acidic, hydroxyl, and quaternary ammonium groups. The α,β-unsaturated compound is selected from acrylate, alkyl acrylate, α,β-unsaturated ester, acrylic acid, acrylamide, vinyl ketone, vinyl sulfone, vinyl phosphonate, and acrylonitrile. Specific examples of the modifying compounds include [2-(acryloyloxy)ethyl]trimethylammonium salt, (3-acrylamidopropyl)trimethylammonium salt, 2-carboxyethyl acrylate, acrylic acid, 2-acrylamido-2-methyl-1-propanesulfonic acid, 3-sulfopropyl acrylate salt, acidic acrylate oligomer, glycidyl trimethylammonium salt, or combinations thereof.

The delivery particles have a modified chitosan content of at least 18 wt % or even at least 21 wt % based on the weight of the shell.

The polyisocyanate useful in the process for forming the polymeric shell is selected from the group consisting of a polyisocyanurate of toluene diisocyanate, a trimethylol propane adduct of toluene diisocyanate, a trimethylol propane adduct of xylylene diisocyanate, 2,2′-methylenediphenyl diisocyanate, 4,4′-methylenediphenyl diisocyanate, 2,4′-methylenediphenyl diisocyanate, [diisocyanato(phenyl)methyl]benzene, toluene diisocyanate, tetramethylxylidene diisocyanate, naphthalene-1,5-diisocyanate, 1,4-phenylene diisocyanate, 1,3-diisocyanatobenzene, derivatives thereof, and combinations thereof.

In an embodiment, the invention described herein teaches a process of forming a population of delivery particles, the delivery particles comprising a core and a shell surrounding the core, the core comprising a benefit agent and an oil phase, wherein the shell comprises a reaction product of at least one modified chitosan and at least one polyisocyanate, the modified chitosan containing a cationic, anionic, or nonionic group covalently bonded to the modified chitosan, the process comprising dissolving chitosan into a water phase at a pH of at least 6.5 or less, the chitosan having amine moieties. Combined into the water phase are a modifying compound containing a reactive group that can form a C—N covalent bond with the amine moieties of the chitosan.

The temperature of the water phase is adjusted to 25° C. or greater, to thereby form a modified chitosan. Optionally, the pH of the modified chitosan solution is adjusted to pH 6.0 or higher. The modifying compound is covalently bonded through C—N bonds with the primary or secondary amine moieties of the chitosan and helps to maintain the modified chitosan dissolved in the water phase even at high pH.

An oil phase is provided, comprising dissolving together at least one benefit agent comprising an oil, and at least one polyisocyanate, optionally with a second oil.

An emulsion is formed by mixing under high shear agitation the oil phase into the water phase, thereby forming droplets of the oil phase and benefit agent dispersed in the water phase. The emulsion is heated to at least 40° C., for a time sufficient to form the shell at an interface of the droplets with the water phase, the shell surrounding the core.

In embodiments, the invention teaches a composition comprising a core-shell delivery particle, the core comprising a benefit agent, the shell comprising a polymer comprising the reaction product of a modified chitosan and a polyisocyanate. The modified chitosan comprises the reaction product of chitosan and a modifying compound. The core comprises a benefit agent and optionally an oil. The modifying compound is selected from epoxide, aldehyde, α,β-unsaturated compound, which is cationic, anionic, or nonionic, and preferably the modifying compound containing an acidic, hydroxyl, or quaternary ammonium group. The α,β-unsaturated compound is selected from acrylate, alkyl acrylate, α,β-unsaturated ester, acrylic acid, acrylamide, vinyl ketone, vinyl sulfone, vinyl phosphonate, and acrylonitrile. Specific examples of the modifying compounds include [2-(acryloyloxy)ethyl]trimethylammonium salt, (3-acrylamidopropyl)trimethylammonium salt, 2-carboxyethyl acrylate, acrylic acid, 2-acrylamido-2-methyl-1-propanesulfonic acid, 3-sulfopropyl acrylate salt, acidic acrylate oligomer, glycidyl trimethylammonium salt, or combinations thereof. In embodiments, at least 21 wt % of the shell is comprised of the modified chitosan, and the shell degrades at least 40% when tested according to test method OECD 301B.

In embodiments the shell comprises 1 to 25 percent by weight of the core-shell delivery particles. The shell degrades at least 40% after at least 60 days, or in some embodiments even after at least 28 days when tested according to test method OECD 301B. The core-shell delivery particles have a ratio of core to shell up to 99:1, or even 99.5:0.5, on the basis of weight.

The benefit agent can be selected from the group consisting of perfume, fragrance, agricultural active, phase change material, essential oil, lubricant, colorant, preservative, antimicrobial active, antifungal active, herbicide, antiviral active, antiseptic active, antioxidant, biological active, deodorant, emollient, humectant, exfoliant, ultraviolet absorbing agent, corrosion inhibitor, silicone oil, wax, bleach particle, fabric conditioner, malodor reducing agent, dye, optical brightener, antiperspirant active and mixtures thereof.

The core-shell delivery particles have a median particle size of from 1 to 200 microns, and the microcapsule is cationic or anionic.

In embodiments the delivery particles have surface charge by virtue of charged domains or charged pendant groups from the composition and process of the invention. Surface charge of the shell is most effectively achieved when the modifying compound has cationic or anionic groups. In particular examples the delivery particles have a zeta potential of 150 mV or less at a pH of 4.5. See for example FIG. 1 herein. As the examples show, the invention enables the zeta potential to be tailored. The invention effects lowering or moderating of the zeta potential at pH conditions of use, yielding a more controllable encapsulate, which usefully is less prone to agglomeration and more compatible with matrices in end use applications

In embodiments the shell degrades at least 60% of its mass after at least 60 days when tested according to test method OECD 301B.

The invention describes a composition and process of forming a population of delivery particles comprising a core and a shell surrounding the core, the process comprising hydrolyzing chitosan by dissolving or dispersing in an acidic medium at a pH of 6.5 or less and a temperature of at least 25° C. A water phase of the hydrolyzed or acid treated chitosan is formed by the above process. The chitosan is further modified by reaction with an epoxide, an aldehyde or an α,β-unsaturated compound. Unlike other processes relying on chitosan the present invention employs a modified chitosan to form a novel multifunctional nucleophile. The modified chitosan is further reactive with a multifunctional electrophile.

In addition, an oil phase is formed by dissolving or dispersing at least one benefit agent and at least one multifunctional electrophile, such as a polyisocyanate, into an oil phase. The benefit agent often can itself be the oil of the oil phase, with the polyisocyanate and benefit agent dissolved together, or optionally with an added oil. An emulsion is formed by mixing, under high shear agitation, the water phase, and the oil phase into an excess of the water phase, thereby forming droplets of the oil phase and benefit agent dispersed in the water phase, with the droplets comprising the core of the core-shell delivery particle. Optionally, the pH of the emulsion can be adjusted in a range from pH 3 to pH 10 or above. A modifying compound comprising an epoxide, an aldehyde or an α,β-unsaturated compound containing acidic, hydroxyl or quaternary ammonium groups is added to the emulsion or water phase.

The emulsion is then cured by heating to at least 40° C., or even at least 60°, for a time sufficient to form a shell at an interface of the droplets with the water phase. The shell is a polymeric material comprising the reaction product of the polyisocyanate and modified chitosan, the shell surrounding the droplets of the oil phase and benefit agent. For many applications, a target droplet size is 0.1 to 100 microns, or even 0.5 to 50 microns. Optionally, curing can also be accomplished by actinic radiation with addition of a UV initiator.

In a further embodiment, to dissolve or disperse the chitosan, the chitosan is processed by being hydrolyzed at a pH of less than 6.5, such as a pH of from pH 3 to pH 6, and a temperature of at least 25° C., or even at least 60° C., or even at least 80° C. The hydrolysis time, depending on pH and temperature can be brief, but more typically would be at least one hour, or even for at least 24 hours. The chitosan is deacetylated to at least 50% or even at least 75%, or even to at least 80%, or even to at least 85%, or even to at least 92%. Desirably, the chitosan has an average size of 600 kilodaltons (kDa) or less. The chitosan is acid treated or hydrolyzed, the expressions “acid treated” or “hydrolyzed” being used interchangeably herein. More importantly the chitosan is modified by reacting with a modifying compound comprising an epoxide, an aldehyde or an α,β-unsaturated compound. The shell formed is a polyurea and the reaction product of polyisocyanate comprising any of isocyanate monomer, oligomer or prepolymer and the modified chitosan. The population of delivery particles can comprise an aqueous slurry, or alternatively can be sprayed onto a substrate, or alternatively spray-dried, resulting in a polyurea-chitosan shell with further chitosan deposited on the surface of the formed delivery particles. The unreacted chitosan in the aqueous slurry, if not decanted, can form the further chitosan deposited on the surface of the formed microcapsules.

In one embodiment, the delivery particles are dried and fracture upon drying, thereby releasing the core. This embodiment can find uses in cleaners with fragrance delivery or in agriculture with a benefit agent such as an agricultural active. Dry-pop type capsules, which fracture on drying, are formed through controlling reaction conditions such as controlling cure time and controlling temperature to yield capsules with thinner walls. Higher cure temperatures, along with longer cure times, can promote higher crosslinking density and enhanced brittleness. A thinner wall, such as from 0.1 nanometer to about 300 nanometers, tends to lend itself to becoming brittle on drying. Even in the dry-pop embodiment, the capsules of the invention exhibit lower leakage and better retention of the core in the capsule slurry pre-drying.

In certain embodiments the modified chitosan in the polyurea shell can be in a range from 18 wt %, or from 21 wt % up to 85 wt % or even to 90 wt % of the total shell as compared to the amount of the electrophile such as polyisocyanate.

In a particular embodiment the process of the invention makes possible a polyurea shell of the core-shell microcapsule having modified chitosan in the polyurea shell (as compared to the amount of polyisocyanate) at 18 wt % or even greater, more particularly 21 wt % to 90 wt %, or even from 21 wt % to 85 wt %, or even 21 wt % to 75 wt %, or 21 wt % to 55 wt % of the total shell being modified chitosan.

The modified chitosan polyurea capsules of the invention in an alternative embodiment make possible forming a reacted polymer shell having a high proportion of modified chitosan moieties in the polymer. Such high weight percent proportions of modified chitosan in a modified chitosan polyurea microcapsule make possible an improved capsule system not previously achieved with interfacial type of encapsulation processes. The process and composition of the invention differ from ionic type of processes based on coacervation, as the polymer of the invention is covalently cross-linked with the polyurea constituent monomers, oligomers and prepolymers forming the modified chitosan polyurea polymer shell.

The composition comprises a core-shell microcapsule, the core comprising a benefit agent, the shell comprising a polyurea resin formed by the reaction of an isocyanate monomer, oligomer or prepolymer and a modified chitosan. The chitosan is first hydrolyzed in an acidic medium at a pH of 6.5 or less and a temperature of at least 25° C., typically for at least one hour.

At low pH, the free amine in chitosan becomes protonated. Chitosan, for purposes hereof, is intended to encompass monomers, oligomers, prepolymers and polymers thereof. When chitosan becomes protonated, conventional understanding would be that chitosan loses the capability of acting as a cross-linker. Chitosan also ceases to act as an emulsifier at low pH, generally of less than pH 4.

Capsules formed, according to the invention exhibit low leakage at high wt % modified chitosan to urea (or polyisocyanate) ratios, and such capsules exhibit degradable properties in relatively short time periods. The capsules additionally are compatible with consumer product matrices such as fabric enhancer, laundry detergent, or similar matrices. Moreover the capsules of the invention exhibit ionic or cationic surface charge. Delivery particles according to the invention are degradable as compared to capsules formed of the same or similar materials under different reaction conditions. Small differences in reaction conditions unexpectedly give rise to encapsulates with significantly different properties. Degradability was found to increase as pH of hydrolysis or acid treatment is decreased below pH 6.5, or even below pH 6 followed by modification with the modifying compound.

Chitosan in the capsule formation process of the invention is dissolved or dispersed, under acidic conditions pH 6.5 or less. The modified chitosan can be formed either during the first hydrolysis/dissolving stage or after the hydrolysis stage, by treatment with a modifying compound. The modified chitosan is soluble at pH 4 or above, or even soluble at pH 6.5 or above.

The delivery particles according to the invention can be fashioned to have a zeta potential up to 15 millivolts (mV) at a pH of 4.5, or even up to 40 mV at a pH of 4.5, or even at least 60 mV at a pH of 4.5, or more preferably 150 mV or less at a pH of 4.5. Such delivery particles can be cationic or anionic and can be useful in applications where deposition onto certain surfaces is desirable. The capsules can be made nonionic or anionic through selection of the modifying compound and the pH.

In one embodiment, the ratio of the isocyanate monomer, oligomer or prepolymer to hydrolyzed chitosan is up to 1:10 by weight. Chitosan as a percentage by weight of the polyisocyanate shell can be as little as 21% up to 95% of the shell. Based on total delivery particles weight, the shell can comprise at least 5% by weight of the core-shell microcapsule, or even at least 3% by weight, or even at least 1% by weight of the core-shell microcapsule, and up to 30% by weight of the core-shell microcapsule. The chitosan can have a degree of deacetylation of at least 75% or even at least 85%, or even at least 92%. The core-shell microcapsule, in certain embodiments, can have a ratio of core to shell up to about 99:1, or even 99.5:0.5 on the basis of weight. The benefit agent of the core-shell delivery particles can be selected from a fragrance, an agricultural active, a phase change material and other actives as described herein. The core-shell delivery particles typically have a median particle size of from 1 to 200 microns. Different particle sizes are obtainable by controlling droplet size during emulsification.

### BRIEF DESCRIPTION OF THE DRAWINGS

FIG. 1 is a graph of zeta potential illustrating delivery particles prepared according to the invention.

### DETAILED DESCRIPTION

The present invention teaches an improved polyurea chitosan microcapsule. In the invention a polyurea microcapsule is successfully prepared by creating a water solution of a hydrolyzed or acid-treated chitosan and modifying the chitosan with a modifying compound. The modified chitosan is a nucleophile and is utilized as a cross-linker to form the shell of a core-shell microcapsule by cross-linking with an electrophile.

In the invention, the modifying compound and chitosan are added into water in a jacketed reactor and at pH from 3 to 6.5, adjusted using acid such as concentrated HCl. The modifying compound and chitosan can also be added into water in a jacketed reactor at high pH, such as pH above 6.5, or even above 8, without pH adjustment. The chitosan mixture is heated to elevated temperature, such as 85° C. in 60 minutes, and then held at this temperature for a time sufficient to effect modification of the chitosan with the modifying compound. The modified chitosan solution is then cooled to 25° C.

The modified chitosan can be prepared from chitosan such as hydrolyzed chitosan in solution or dispersed chitosan by treatment with a modifying compound. The chitosan is further modified by reaction with an epoxide, an aldehyde or an α,β-unsaturated compound. Unlike other processes relying on chitosan, the present invention employs a modified chitosan to form a novel multifunctional nucleophile. The modified chitosan is further reactive with a multifunctional electrophile.

The modified compound for modifying chitosan can be selected from epoxides such as: glycidyl trimethylammonium salt, glycidyl isopropyl ether, glycidyl methacrylate, furfuryl glycidyl ether, glycidol, 1,4-butanediol diglycidyl ether, 2-ethylhexyl glycidyl ether, (3-glycidyloxypropyl) trimethoxysilane, poly(ethylene glycol) diglycidyl ether, trimethylolpropane triglycidyl ether or aldehydes such as glutaraldehyde, Alginate Aldehyde, or α,β-unsaturated compounds such as acrylic acid, acrylate salt, maleic acid, vinyl sulfonic acid, 2-carboxyethyl acrylate, 2-(2-oxo-1-imidazolidinyl)ethyl methacrylate, 2-(2-oxo-1-imidazolidinyl)ethyl methacrylamide, (2-(acryloyloxy)ethyl)trimethylammonium salt, (3-(methacryloylamino)propyl) trimethylammonium salt, N,N-dialkylaminoalkyl acrylate, N,N-dialkylaminoalkyl acrylamide, (3-acrylamidopropyl)trimethylammonium salt, 3-sulfopropyl acrylate salt, 2-acrylamido-2-methyl-1-propanesulfonic acid and their salts, quaternized vinyl imidazole, diallyl dialkyl ammonium salts, vinyl amine.

An oil phase is prepared by dissolving an electrophile, for example, an isocyanate such as trimers of xylylene Diisocyanate (XDI) or polymers of methylene diphenyl diisocyanate (MDI), in oil at 25° C. Diluents, for example isopropyl myristate, may be used to adjust the hydrophilicity of the oil phase. The oil phase is then added into the water phase and milled at high speed to obtain a targeted size. The emulsion is then cured in one or more heating steps, such as heating to 40° C. in 30 minutes and holding at 40° C. for 60 minutes. Times and temperatures are approximate. The temperature and time are selected to be sufficient to form and cure a shell at the interface of the droplets of the oil phase with the water continuous phase. For example, the emulsion is heated to 85° C. in 60 minutes and then held at 85° C. for 360 minutes to cure the capsules. The slurry is then cooled to room temperature. Optionally, the same modifying compound or a different modifying compound is instead added to the emulsion to further modify the chitosan.

The electrophile useful in the invention is to be understood for purposes hereof as a polyisocyanate such as isocyanate monomer, isocyanate oligomer, isocyanate prepolymer, or dimer or trimer of an aliphatic or aromatic isocyanate. Useful polyisocyanates. contains two or more isocyanate (—NCO) groups. These polyisocyanate electrophiles can be aromatic, aliphatic, linear, branched, or cyclic. All such electrophiles, whether monomers, prepolymers, oligomers, or dimers or trimers of aliphatic or aromatic isocyanates are intended encompassed by the term “polyisocyanate” herein.

The polyisocyanate is an aliphatic or aromatic monomer, oligomer or prepolymer, usefully of two or more isocyanate functional groups. The polyisocyanate, for example, can be selected from aromatic toluene diisocyanate and its derivatives used in wall formation for encapsulates, or aliphatic monomer, oligomer or prepolymer, for example, hexamethylene diisocyanate and dimers or trimers thereof, or 3,3,5-trimethyl-5-isocyanatomethyl-1-isocyanato cyclohexane tetramethylene diisocyanate. The polyisocyanate can be selected from 1,3-diisocyanato-2-methylbenzene, hydrogenated MDI, bis(4-isocyanatocyclohexyl)methane, dicyclohexylmethane-4,4′-diisocyanate, and oligomers and prepolymers thereof. This listing is illustrative and not intended to be limiting of the polyisocyanates useful in the invention.

The polyisocyanates useful in the invention comprise isocyanate monomers, oligomers or prepolymers, or dimers or trimers thereof, having at least two isocyanate groups. Optimal cross-linking can be achieved with polyisocyanates having at least three functional groups.

Electrophiles useful in the invention include polyisocyanates. Polyisocyanates, for purposes of the invention, are understood as encompassing any polyisocyanate having at least two isocyanate groups and comprising an aliphatic or aromatic moiety in the monomer, oligomer or prepolymer. Aromatic polyisocyanate is understood as a polyisocyanate which comprises at least one aromatic moiety. If aromatic, the aromatic moiety can comprise a phenyl, a toluyl, a xylyl, a naphthyl or a diphenyl moiety, more preferably a toluyl or a xylyl moiety. Aromatic polyisocyanates, for purposes hereof, can include diisocyanate derivatives such as biurets and polyisocyanurates. The polyisocyanate, when aromatic, can be, but is not limited to, methylene diphenyl diisocyanate, toluene diisocyanate, tetramethylxylidene diisocyanate, polyisocyanurate of toluene diisocyanate (commercially available from Bayer under the tradename Desmodur® RC), trimethylol propane-adduct of toluene diisocyanate (commercially available from Bayer under the tradename Desmodur® L75), or naphthalene-1,5-diisocyanate, and phenylene diisocyanate, or trimethylol propane-adduct of xylylene diisocyanate (commercially available from Mitsui Chemicals under the tradename Takenate® D-110N).

Polyisocyanate, which is aliphatic, is understood as a polyisocyanate which does not comprise any aromatic moiety. Aromatic polyisocyanate is understood as a polyisocyanate which comprises at least one aromatic moiety. There is a preference for aromatic polyisocyanate, however, aliphatic polyisocyanates and blends thereof are useful. Aliphatic polyisocyanates include a trimer of hexamethylene diisocyanate, a trimer of isophorone diisocyanate, a trimethylol propane-adduct of hexamethylene diisocyanate (available from Mitsui Chemicals) or a biuret of hexamethylene diisocyanate (commercially available from Bayer under the tradename Desmodur® N 100).

Electrophiles need not be limited to polyisocyanates. Electrophiles can comprise monomers, oligomers and prepolymers having electrophilic moieties and such electrophilic moieties can include any of formyl, keto, carboxyl, isocyanate, carboxylate ester, acyl halide, amides carboxylic anhydride, alkyl halide, epoxide, sulfonyl halide, chlorophosphate, β-unsaturated carbonyl, α,β-unsaturated nitrile, trifluoromethanesulfonate, p-toluenesulfonate, and α,β-unsaturated methanesulfonyl groups.

Suitable polyfunctional electrophiles can include glutaric dialdehyde, succinic dialdehyde, glyoxal; glyoxyl trimer, paraformaldehyde, bis(dimethyl) acetal, bis(diethyl) acetal, polymeric dialdehydes, such as oxidized starch, low molecular weight difunctional aldehydes, 1,3-propane dialdehyde, 1,4-butane dialdehyde, 1,5-pentane dialdehyde, or 1,6-hexane.

The capsule shell could also be reinforced using additional co-crosslinkers such as multifunctional amines and/or polyamines such as diethylene triamine (DETA), polyethylene imine, and polyvinyl amine.

### Core

The delivery particles of the present teaching include a benefit agent which comprises one or more ingredients that are intended to be encapsulated. The benefit agent is selected from a number of different materials such as chromogens and dyes, flavorants, perfumes, sweeteners, fragrances, oils, fats, pigments, cleaning oils, pharmaceuticals, pharmaceutical oils, perfume oils, mold inhibitors, antimicrobial agents, adhesives, phase change materials, scents, fertilizers, nutrients, and herbicides: by way of illustration and without limitation. The benefit agent and oil comprise the core. The core can be a liquid or a solid. With cores that are solid at ambient temperatures, the wall material can usefully enwrap less than the entire core for certain applications where availability of, for example, an agglomerate core is desired on application. Such uses can include scent release, cleaning compositions, emollients, cosmetic delivery, and the like. Where the microcapsule core is phase change material, uses can include such encapsulated materials in mattresses, pillows, bedding, textiles, sporting equipment, medical devices, building products, construction products, HVAC, renewable energy, clothing, athletic surfaces, electronics, automotive, aviation, shoes, beauty care, laundry, and solar energy.

The core constitutes the material encapsulated by the delivery particles. Typically, particularly when the core material is a liquid material, the core material is combined with one or more of the compositions from which the internal wall of the microcapsule is formed or solvent for the benefit agent or partitioning modifier. If the core material can function as the oil solvent in the capsules, e.g. acts as the solvent or carrier for either the wall forming materials or benefit agent, it is possible to make the core material the major material encapsulated, or if the carrier itself is the benefit agent, can be the total material encapsulated. Usually however, the benefit agent is from 0.01 to 99 weight percent of the capsule internal contents, preferably 0.01 to about 65 by weight of the capsule internal contents, and more preferably from 0.1 to about 45% by weight of the capsule internal contents. With certain applications, the core material can be effective even at just trace quantities.

Where the benefit agent is not itself sufficient to serve as the oil phase or solvent, particularly for the wall forming materials, the oil phase can comprise a suitable carrier and/or solvent. In this sense, the oil is optional, as the benefit agent itself can at times be the oil. These carriers or solvents are generally an oil, preferably have a boiling point greater than about 80° C. and low volatility and are non-flammable. Though not limited thereto, they preferably comprise one or more esters, preferably with chain lengths of up to 18 carbon atoms or even up to 42 carbon atoms and/or triglycerides such as the esters of C6 to C12 fatty acids and glycerol. Exemplary carriers and solvents include, but are not limited to: ethyldiphenylmethane; isopropyl diphenylethane; butyl biphenyl ethane; benzylxylene; alkyl biphenyls such as propylbiphenyl and butylbiphenyl; dialkyl phthalates e.g. dibutyl phthalate, dioctylphthalate, dinonyl phthalate and ditridecylphthalate; 2,2,4-trimethyl-1,3-pentanediol diisobutyrate; alkyl benzenes such as dodecyl benzene; alkyl or aralkyl benzoates such as benzyl benzoate; diaryl ethers; di(aralkyl)ethers and aryl aralkyl ethers; ethers such as diphenyl ether, dibenzyl ether and phenyl benzyl ether; liquid higher alkyl ketones (having at least 9 carbon atoms); alkyl or aralkyl benzoates, e.g., benzyl benzoate; alkylated naphthalenes such as dipropylnaphthalene; partially hydrogenated terphenyls; high-boiling straight or branched chain hydrocarbons; alkaryl hydrocarbons such as toluene; vegetable and other crop oils such as canola oil, soybean oil, corn oil, sunflower oil, cottonseed oil, lemon oil, olive oil and pine oil; methyl esters of fatty acids derived from transesterification of vegetable and other crop oils, methyl ester of oleic acid, esters of vegetable oil, e.g. soybean methyl ester, straight chain paraffinic aliphatic hydrocarbons, and mixtures of the foregoing.

Useful benefit agents include perfume raw materials, such as alcohols, ketones, aldehydes, esters, ethers, nitriles, alkenes, fragrances, fragrance solubilizers, essential oils, phase change materials, lubricants, colorants, cooling agents, preservatives, antimicrobial or antifungal actives, herbicides, antiviral actives, antiseptic actives, antioxidants, biological actives, deodorants, emollients, humectants, exfoliants, ultraviolet absorbing agents, self-healing compositions, corrosion inhibitors, sunscreens, silicone oils, waxes, hydrocarbons, higher fatty acids, essential oils, lipids, skin coolants, vitamins, sunscreens, antioxidants, glycerine, catalysts, bleach particles, silicon dioxide particles, malodor reducing agents, dyes, brighteners, antibacterial actives, antiperspirant actives, cationic polymers and mixtures thereof. Phase change materials useful as benefit agents can include, by way of illustration and not limitation, paraffinic hydrocarbons having 13 to 28 carbon atoms, various hydrocarbons such n-octacosane, n-heptacosane, n-hexacosane, n-pentacosane, n-tetracosane, n-tricosane, n-docosane, n-heneicosane, n-eicosane, n-nonadecane, octadecane, n-heptadecane, n-hexadecane, n-pentadecane, n-tetradecane, n-tridecane. Phase change materials can alternatively, optionally in addition include crystalline materials such as 2,2-dimethyl-1,3-propanediol, 2-hydroxymethyl-2-methyl-1,3-propanediol, acids of straight or branched chain hydrocarbons such as eicosanoic acid and esters such as methyl palmitate, fatty alcohols, and mixtures thereof.

Preferably, in the case of fragrances, a perfume oil acts as benefit agent and solvent for the wall forming material, as illustrated in the examples herein.

The invention makes possible tailored surface charge of chitosan urea-based delivery particles by chemical attachment on the surface, especially the external surface of the microcapsule, through the charged domains or charged pendant groups of the resulting polymer.

Optionally the water phase may include an emulsifier. Non-limiting examples of emulsifiers include water-soluble salts of alkyl sulfates, alkyl ether sulfates, alkyl isothionates, alkyl carboxylates, alkyl sulfosuccinates, alkyl succinamates, alkyl sulfate salts such as sodium dodecyl sulfate, alkyl sarcosinates, alkyl derivatives of protein hydrolyzates, acyl aspartates, alkyl or alkyl ether or alkylaryl ether phosphate esters, sodium dodecyl sulphate, phospholipids or lecithin, or soaps, sodium, potassium or ammonium stearate, oleate or palmitate, alkylarylsulfonic acid salts such as sodium dodecylbenzenesulfonate, sodium dialkylsulfosuccinates, dioctyl sulfosuccinate, sodium dilaurylsulfosuccinate, poly(styrene sulfonate) sodium salt, isobutylene-maleic anhydride copolymer, gum arabic, sodium alginate, carboxymethylcellulose, cellulose sulfate and pectin, poly(styrene sulfonate), isobutylene-maleic anhydride copolymer, carrageenan, sodium alginate, pectic acid, tragacanth gum, almond gum and agar; semi-synthetic polymers such as carboxymethyl cellulose, sulfated cellulose, sulfated methylcellulose, carboxymethyl starch, phosphated starch, lignin sulfonic acid; and synthetic polymers such as maleic anhydride copolymers (including hydrolyzates thereof), polyacrylic acid, polymethacrylic acid, acrylic acid butyl acrylate copolymer or crotonic acid homopolymers and copolymers, vinyl benzenesulfonic acid or 2-acrylamido-2-methylpropanesulfonic acid homopolymers and copolymers, and partial amide or partial ester of such polymers and copolymers, carboxy modified polyvinyl alcohol, sulfonic acid-modified polyvinyl alcohol and phosphoric acid-modified polyvinyl alcohol, phosphated or sulfated tristyrylphenol ethoxylates, palmitamidopropyltrimonium chloride (Varisoft PATC™, available from Degussa Evonik, Essen, Germany), distearyl diammonium chloride, cetyltrimethylammonium chloride, quaternary ammonium compounds, fatty amines, aliphatic ammonium halides, alkyldimethylbenzylammonium halides, alkyldimethylethylammonium halides, polyethyleneimine, poly(2-dimethylamino)ethyl methacrylate) methyl chloride quaternary salt, poly(l-vinylpyrrolidone-co-2-dimethylaminoethyl methacrylate), poly(acrylamide-co-diallyldimethylammonium chloride), poly(allylamine), poly[bis(2-chloroethyl) ether-alt-1,3-bis[3-(dimethylamino)propyl]urea] quaternized, and poly(dimethylamine-co-epichlorohydrin-co-ethylenediamine), condensation products of aliphatic amines with alkylene oxide, quaternary ammonium compounds with a long-chain aliphatic radical, e.g. distearyldiammonium chloride, and fatty amines, alkyldimethylbenzylammonium halides, alkyldimethylethylammonium halides, polyalkylene glycol ether, condensation products of alkyl phenols, aliphatic alcohols, or fatty acids with alkylene oxide, ethoxylated alkyl phenols, ethoxylated aryl phenols, ethoxylated polyaryl phenols, carboxylic esters solubilized with a polyol, polyvinyl alcohol, polyvinyl acetate, or copolymers of polyvinyl alcohol polyvinyl acetate, polyacrylamide, poly(N-isopropylacrylamide), poly(2-hydroxypropyl methacrylate), poly(-ethyl-2-oxazoline), poly(2-isopropenyl-2-oxazoline-co-methyl methacrylate), poly(methyl vinyl ether), and polyvinyl alcohol-co-ethylene), and cocoamidopropyl betaine. Emulsifier, if employed, is typically from about 0.1 to 40% by weight, preferably 0.2 to about 15% by weight, more typically 0.5 to 10% be weight, based on total weight of the formulation

The delivery particles may encapsulate a partitioning modifier in addition to the benefit agent. Non-limiting examples of partitioning modifiers include isopropyl myristate, mono-, di-, and tri-esters of C₄-C₂₄ fatty acids, castor oil, mineral oil, soybean oil, hexadecanoic acid, methyl ester isododecane, isoparaffin oil, polydimethylsiloxane, brominated vegetable oil, and combinations thereof. Delivery particles may also have varying ratios of the partitioning modifier to the benefit agent so as to make different populations of delivery particles that may have different bloom patterns. Such populations may also incorporate different perfume oils so as to make populations of delivery particles that display different bloom patterns and different scent experiences. US 2011-0268802 discloses other non-limiting examples of delivery particles and partitioning modifiers and is hereby incorporated by reference.

In the formation of the chitosan delivery particles, the aqueous solution contains a residual quantity of the hydrolyzed chitosan. This provides the option of dewatering the delivery particles such as through decanting, filtration, centrifuging, or other separation technique. Alternatively, the aqueous slurry of chitosan polyurea delivery particles can be spray dried forming chitosan polyurea delivery particles further coated with a layer of the residual hydrolyzed chitosan from the water phase.

In one embodiment, the formed slurry of delivery particles can be further dispersed in additional water or with low concentration of residual overcoating hydrolyzed chitosan yielding chitosan polyurea delivery particles that can fracture upon drying, providing an additional release mechanism useful in some applications such as fragrance delivery or with agricultural actives for targeted delivery.

In some examples of the process and compositions, the delivery particles may consist of one or more distinct populations. The composition may have at least two different populations of delivery particles that vary in the exact make-up of the perfume oil and in the median particle size and/or partitioning modifier to perfume oil (PM:PO) weight ratio. In some examples, the composition includes more than two distinct populations that vary in the exact make up the perfume oil and in their fracture strengths. In some further examples, the populations of delivery particles can vary with respect to the weight ratio of the partitioning modifier to the perfume oil(s). In some examples, the composition can include a first population of delivery particles having a first ratio that is a weight ratio of from 2:3 to 3:2 of the partitioning modifier to a first perfume oil and a second population of delivery particles having a second ratio that is a weight ratio of less than 2:3 but greater than 0 of the partitioning modifier to a second perfume oil.

In some embodiments, each distinct population of delivery particles is preparable in a distinct slurry. For example, the first population of delivery particles can be contained in a first slurry and the second population of delivery particles contained in a second slurry. It is to be appreciated that the number of distinct slurries for combination is without limit and a choice of the formulator such that 3, 10, or 15 distinct slurries may be combined. The first and second populations of delivery particles may vary in the exact make up the perfume oil and in the median particle size and/or PM:PO weight ratio.

In some embodiments, the composition, can be prepared by combining the first and second slurries with at least one adjunct ingredient and optionally packaged in a container. In some examples, the first and second populations of delivery particles can be prepared in distinct slurries and then spray dried to form a particulate. The distinct slurries may be combined before spray drying, or spray dried individually and then combined together when in particulate powder form. Once in powder form, the first and second populations of delivery particles may be combined with an adjunct ingredient to form the composition useful as a feedstock for manufacture of consumer, industrial, medical, or other goods. In some examples, at least one population of delivery particles is spray dried and combined with a slurry of a second population of delivery particles. In some examples, at least one population of delivery particles is dried, prepared by spray drying, fluid bed drying, tray drying, or other such drying processes that are available.

In some examples, the slurry or dry particulates can include one or more adjunct materials such as processing aids selected from the group consisting of a carrier, an aggregate inhibiting material, a deposition aid, a particle suspending polymer, and mixtures thereof. Non-limiting examples of aggregate inhibiting materials include salts that can have a charge-shielding effect around the particle, such as magnesium chloride, calcium chloride, magnesium bromide, magnesium sulfate, and mixtures thereof. Non-limiting examples of particle suspending polymers include polymers such as xanthan gum, carrageenan gum, guar gum, shellac, alginates, chitosan; cellulosic materials such as carboxymethyl cellulose, hydroxypropyl methyl cellulose, cationically charged cellulosic materials; polyacrylic acid; polyvinyl alcohol; hydrogenated castor oil; ethylene glycol distearate; and mixtures thereof.

In some embodiments, the slurry can include one or more processing aids, selected from the group consisting of water, aggregate inhibiting materials such as divalent salts; particle suspending polymers such as xanthan gum, guar gum, carboxy methyl cellulose.

In other examples of the invention, the slurry can include one or more carriers selected from the group consisting of polar solvents, including but not limited to, water, ethylene glycol, propylene glycol, polyethylene glycol, glycerol; nonpolar solvents, including but not limited to, mineral oil, perfume raw materials, silicone oils, hydrocarbon paraffin oils, and mixtures thereof.

In some examples, said slurry may include a deposition aid that may comprise a polymer selected from the group comprising: polysaccharides, in one aspect, cationically modified starch and/or cationically modified guar; polysiloxanes; poly diallyl dimethyl ammonium halides; copolymers of poly diallyl dimethyl ammonium chloride and polyvinyl pyrrolidone; a composition comprising polyethylene glycol and polyvinyl pyrrolidone; acrylamides; imidazoles; imidazolinium halides; polyvinyl amine; copolymers of poly vinyl amine and N-vinyl formamide; polyvinyl formamide, polyvinyl alcohol; polyvinyl alcohol crosslinked with boric acid; polyacrylic acid; polyglycerol ether silicone cross-polymers; polyacrylic acids, polyacrylates, copolymers of polyvinylamine and polyvinylalcohol oligomers of amines, in one aspect a diethylenetriamine, ethylene diamine, bis(3-aminopropyl)piperazine, N,N-Bis-(3-aminopropyl)methylamine, tris(2-aminoethyl)amine and mixtures thereof; polyethyleneimine, a derivatized polyethyleneimine, in one aspect an ethoxylated polyethyleneimine; a polymeric compound comprising, at least two moieties selected from the moieties consisting of a carboxylic acid moiety, an amine moiety, a hydroxyl moiety, and a nitrile moiety on a backbone of polybutadiene, polyisoprene, polybutadiene/styrene, polybutadiene/acrylonitrile, carboxyl-terminated polybutadiene/acrylonitrile or combinations thereof; pre-formed coacervates of anionic surfactants combined with cationic polymers; polyamines and mixtures thereof.

In some additional examples to illustrate the invention, at least one population of delivery particles can be contained in an agglomerate and then combined with a distinct population of delivery particles and at least one adjunct material. Said agglomerate may comprise materials selected from the group consisting of silicas, citric acid, sodium carbonate, sodium sulfate, sodium chloride, and binders such as sodium silicates, modified celluloses, polyethylene glycols, polyacrylates, polyacrylic acids, zeolites, and mixtures thereof.

Suitable equipment for use in the processes disclosed herein may include continuous stirred tank reactors, homogenizers, turbine agitators, recirculating pumps, paddle mixers, plough shear mixers, ribbon blenders, vertical axis granulators and drum mixers, both in batch and, where available, in continuous process configurations, spray dryers, and extruders. Such equipment can be obtained from Lodige GmbH (Paderborn, Germany), Littleford Day, Inc. (Florence, Ky., U.S.A.), Forberg AS (Larvik, Norway), Glatt Ingenieurtechnik GmbH (Weimar, Germany), Niro (Soeborg, Denmark), Hosokawa Bepex Corp. (Minneapolis, Minn., U.S.A.), Arde Barinco (New Jersey, U.S.A.).

In the process of the invention, chitosan is dissolved or dispersed in water with mixing. Optionally, the pH is adjusted to be acidic, such as pH of 3, 4, 5 or 6. A modifying compound, such as epoxide, aldehyde, or an α,β-unsaturated compound is added to the chitosan in solution. By way of illustration and not limitation such a modifying compound, can be of the α,β-unsaturated type. For example, the modifying compound can be selected to be [2-(acryloyloxy)ethyl]trimethylammonium salt. Other such salts are also described herein and in the examples. The solution is mixed at elevated temperature such as from 65° C., preferably 70° C., or even 90° C. for a period sufficient to effect forming the modified chitosan.

An oil phase is prepared by mixing together a benefit agent such as a fragrance, together with an isocyanate and optionally a partitioning modifier such as isopropyl myristate. An emulsion is formed by adding the oil phase comprising the benefit agent such as fragrance to the water phase under shear to form an emulsion. The emulsion is heated at elevated temperature such as from 65 to 90° C. or even 95° C. for a sufficient period to form a shell at an interface of the droplets with the water phase, the shell surrounding the benefit agent forming the core of the core-shell delivery particles.

In an in situ variation of the process, chitosan is dissolved or dispersed in water to create a chitosan solution. The pH is adjusted to be acidic. An oil phase is prepared by mixing together a benefit agent such as a fragrance, together with an isocyanate and optionally a portioning modifier such as isopropyl myristate. The oil phase is added to the water phase under shear forming an emulsion comprising droplets of the oil phase with benefit agent dispersed in the water phase. In this alternate embodiment of the process, the chitosan is modified in situ by addition of the modifying compound to the emulsion.

The modifying compound such as an epoxide, the aldehyde, or the α,β-unsaturated compound, is illustrated for example in Example 8 employing an acid acrylate oligomer as the α,β-unsaturated compound. The modifying compound is added to the emulsion under mixing and heated such as from 65° C. to 70° C. or even to 90° C. or even 95° C. for a sufficient period to modify the chitosan in situ and form a shell at an interface of the oil phase droplets with the water phase, the shell surrounding the oil droplets containing the benefit agent.

In the process of the invention the dissolved or dispersed chitosan in water is acidified to a pH of 6.5 or less, treated with acid or hydrolyzed with acid, these descriptions of acid treated chitosan or hydrolyzed chitosan to be understood as interchangeable herein. The acid treated or hydrolyzed chitosan is modified with the modifying compound comprising an epoxide, an aldehyde, or an α,β-unsaturated compound.

The modifying compound, such as epoxide, aldehyde, or an α,β-unsaturated compound is added to the chitosan in solution, or in the in-situ variation described earlier, the modifying compound is added to emulsion following addition of the oil phase to the water phase. Optionally, the modifying compound can be a second modifying compound added to the emulsion.

In a further optional variation, a redox initiator comprising a persulfate or a peroxide can be added to the acid treated or hydrolyzed chitosan. In the in-situ variation, a redox initiator can be added to the emulsion following combining of the oil phase and water phase under high shear agitation. The redox initiator advantageously depolymerizes the hydrolyzed chitosan or modified chitosan reducing viscosity facilitating polymer formation of the shell in the capsule formation process. Modification of chitosan with the epoxide, aldehyde, or α,β-unsaturated compound is preferably accomplished first, although the redox initiator (peroxide or persulfate) can be introduced at the same time as the modifying compound or even before. The redox initiator can be selected from the group consisting of ammonium persulfate, sodium persulfate, potassium persulfate cesium persulfate, benzoyl peroxide, and hydrogen peroxide. The ratio of persulfate or peroxide to raw chitosan is from 0.01/99.99 to 95/5 on the basis of weight.

Accordingly, for clarity, in the process there are several variations. The acidified chitosan solution can be treated with the modifying compound by addition of the modifying compound to the chitosan in solution in the first instance. Alternatively, the modifying compound can be added to the emulsion. Similarly and independently, an optional redox initiator can be added to the chitosan in solution which is acidified or added into the emulsion in the emulsification step following addition of the oil phase. The redox initiator can be added before, concurrently, or sequentially after the modification step with the modifying compound comprising epoxide, aldehyde, or an α,β-unsaturated compound.

### Procedure for Determination of % Degradation

% degradation is determined by the “OECD Guideline for Testing of Chemicals” 301B CO₂ Evolution (Modified Sturm Test), adopted 17 Jul. 1992. For ease of reference, this test method is referred to herein as test method OECD 301B

### Procedure for Determination of Free Oil

This method measures the amount of oil in the water phase and uses as an internal standard solution 1 mg/ml dibutyl phthalate (DBP)/hexane.

Weigh a little more than 250 mgs of DBP into a small beaker and transfer to a 250 ml volumetric rinsing the beaker thoroughly. Fill with hexane to 250 ml.

Sample Prep: Weigh approximately 1.5-2 grams (40 drops) of the capsule slurry into a 20 ml scintillation vial and add 10 ml's of the ISTD solution, cap tightly. Shaking vigorously several times over 30 minutes, pipette solution into an autosampler vial and analyze by GC.

Additional details. Instrumentation: HP5890 GC connected to HP Chem Station Software; Column: 5 m×0.32 mm id with 1 μm DB-1 liquid phase; Temperature 50 deg for 1 minute then heat to 320 deg @ 15 deg/min; Injector: 275° C.; Detector: 325° C.; 2 ul injection.

Calculation: Add total peak area minus the area for the DBP for both the sample and calibration. Calculate mg of free core oil:

$$\frac{total area from sample}{total area from calibration} \times mg of oil in calibration solution = mg of free oi$$

Calculate % free core oil

$$\frac{mg of free core oil}{sample wt ( mg )} \times 100 = \% free core oil in wet slurry$$

### Procedure for Determination of Benefit Agent Leakage

Obtain 2, one gram samples of benefit agent particle composition. Add 1 gram (Sample 1) of particle composition to 99 grams of product matrix in which the particle will be employed. Age the particle containing product matrix (Sample 1) for 2 weeks at 35° C. in a sealed glass jar. The other 1 gram sample (Sample 2) is similarly aged.

After 2 weeks, use filtration to recover the particle composition's particles from the product matrix (Sample 1) and from the particle composition (Sample 2). Treat each particle sample with a solvent that will extract all the benefit agent from each samples' particles. Inject the benefit agent containing solvent from each sample into a Gas Chromatograph and integrate the peak areas to determine the total quantity of benefit agent extracted from each sample.

Determine the percentage of benefit agent leakage by calculating the difference in the values obtained for the total quantity of benefit agent extracted from Sample 2 minus Sample 1, expressed as a percentage of the total quantity of benefit agent extracted from Sample 2, as represented in the equation below:

$$( \frac{Sample 2 - Sample 1}{Sample 2} ) \times 100 = Percentage of Benefit Agent Leakage$$

### Procedure for Qualitatively Measuring Compatibility of Delivery Particles in Laundry Matrix

The compatibility of the delivery particles in laundry matrix is measured by visually inspect the mixture of delivery particles and laundry matrix in glass jar. The slurry containing the delivery particles were homogenized by agitation for at least one minutes with an overhead mixer. The homogenized slurry was then added in laundry matrix, such as heavy duty laundry matrix at a ratio of 1:40, such as 1 g slurry in 40 g matrix, under mixing. Mixing the above mixture for at least 15 minutes at 350 rpm using overhead mixer. Stop mixing and let the mixture stand for 5 minutes before examination. Visually inspect the mixture with naked eye and under optical microscope to detect any aggregates in the mixture. If any aggregates observed with naked eye or greater than 100 micron under optical microscope, the delivery particles are determined to be not compatible in laundry matrix.

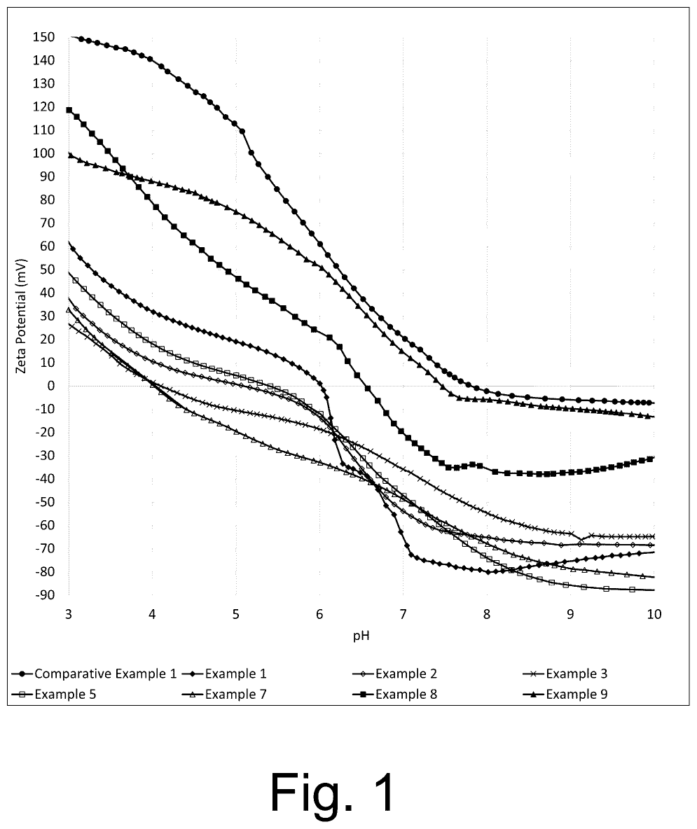

Polyurea capsules prepared with chitosan exhibit positive zeta potentials as shown in FIG. 1. Such capsules have improved deposition efficiency on fabrics.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

In the following examples, the abbreviations correspond to the materials listed in Table 1.

TABLE 1

| Trade | | |
|---|---|---|
| Name | Material | Company/City |
| Selvol 540 | Polyvinyl alcohol | Sekisui Specialty |
| | | Chemicals, Dallas, TX |
| ChitoClear | Chitosan | Primex EHF, Siglufjordur, |
| | | Iceland |
| Takenate | Polyisocyanate prepolymer | Mitsui Chemicals America, |
| D-110N | | Inc., Rye Brook, NY |
| CD9055 | Acidic acrylate oligomer | Arkema Inc., King of |
| | | Prussia, Pennsylvania |
| SR444 | Pentaerythritol triacrylate | Arkema Inc., King of |
| | | Prussia, Pennsylvania |
| SR268 | Tetraethylene glycol diacrylate | Arkema Inc., King of |
| | | Prussia, Pennsylvania |
| Caustic | Sodium hydroxide | Hydrite Chemical Co., |
| soda | | Brookfield, WI |
| HCl | Hydrochloric acid | Avantor Performance |
| | | Materials, LLC, Radnor, |
| | | PA |
| | [2-(Acryloyloxy)ethyl]trimethyl- | Sigma-Aldrich Inc., St. |
| | ammonium chloride solution | Louis, MO |
| | Glacial acetic acid | Avantor Performance |
| | | Materials, LLC, Radnor, |
| | | PA |
| | Isopropyl myristate | Acme-Hardesty Co., Bule |
| | | Bell, PA |
| | Acrylic acid | TCI America, Portland, OR |
| | 3-Sulfopropyl acrylate potassium | Sigma-Aldrich Inc., St. |
| | salt | Louis, MO |
| | Potassium persulfate | Avantor Performance |
| | | Materials, LLC, Radnor, |
| | | PA |

### EXAMPLES

### Comparative Example 1. Polyurea Capsule with Unmodified Chitosan

A chitosan stock solution is prepared by dispersing 39.60 g chitosan Chitoclear into 840.4 g deionized water while mixing in a jacketed reactor. The pH of the chitosan dispersion is then adjusted to 3.87 using 17.90 g concentrated HCl under agitation. The temperature of the chitosan solution is then increased to 85° C. over 60 minutes and then held at 85° C. for 2 hours to hydrolyze the chitosan. The temperature is then reduced to 25° C. after the hydrolyzing step over a period of 90 minutes. The pH of the hydrolyzed chitosan solution is 3.97.

A water phase is prepared by mixing 426.30 g of the above chitosan stock solution and 6.70 g 5% PVA540 solution in a jacketed reactor. An oil phase is prepared by mixing 146.63 g perfume and 36.66 g isopropyl myristate together along with 4.00 g Takenate D-110N at room temperature. The oil phase is added to the water phase under high shear milling to obtain an emulsion with desired particle size. The emulsion is heated to 40° C. in 30 minutes and then hold at 40° C. for 60 minutes. The emulsion is then heated to 85° C. in 60 minutes and maintained at this temperature for 6 hours while mixing. The formed capsules have a median particle size of 8.05 micron. The capsules formed had a one week leakage of 20.78%. The prepared slurry shows aggregation in heavy duty liquid laundry matrix.

### Inventive Examples

### Example 1: Crosslinked Chitosan Delivery Particles Pre-Modified with [2-(Acryloyloxy)Ethyl]Trimethylammonium Chloride

A modified chitosan solution was prepared by dispersing 40.92 g chitosan powder in 792.00 water at 70° C. The pH of the above mixture was adjusted to 4.91 using 9.79 g glacial acetic acid. 46.36 g 80% [2-(Acryloyloxy)ethyl]trimethylammonium chloride solution was then added to the above chitosan solution and mixed at 70° C. for 12 hours to obtain the [2-(Acryloyloxy)ethyl]trimethylammonium chloride modified chitosan solution. The pH of the obtained modified chitosan solution is 4.05.

A water phase is prepared by weighting 255.8 g [2-(Acryloyloxy)ethyl]trimethylammonium chloride modified chitosan solution in jacketed reactor at 25° C.

An oil phase is prepared by mixing 87.98 g perfume, 2.40 g Takenate D110 and 22.00 g Isopropyl myristate at 25° C. in a beaker.

The oil phase is added into the water phase under high shear for a period of time to obtain an emulsion at room temperature.

The emulsion is then heated to 40° C. in 30 minutes and then hold at 40° C. for 60 minutes. The emulsion is then heated to 85° C. in 60 minutes and hold for 6 hours to cure the wall. The emulsion is then cooled down to 25° C. in 90 minutes. The obtained capsule has median particle size of 12.27 micron. The QFO and 1 week leakage of the capsule slurry is 7.69% and 68.59% respectively. The prepared slurry shows no aggregation in heavy duty liquid laundry matrix.

### Example 2: Crosslinked Chitosan Delivery Particles Pre-Modified with [2-(Acryloyloxy)Ethyl]Trimethylammonium Chloride and pH Adjustment

Example 2 is prepared following the procedure of example 1 beside the pH of the water phase was adjusted to 6.8 using sodium hydroxide solution. The water phase is still clear after pH adjustment. The obtained capsule has median particle size of 36.44 micron. The QFO and 1 week leakage of the capsule slurry is 0.11% and 0.99% respectively. The prepared slurry shows no aggregation in heavy duty liquid laundry matrix.

### Example 3: Crosslinked Chitosan Delivery Particles Pre-Modified with [2-(Acryloyloxy)Ethyl]Trimethylammonium Chloride and pH Adjustment with a Second Modifying Compound—SR444

A modified chitosan solution is prepared according to the procedures in Example 1. The modified chitosan solution has pH of 4.10. A water phase is comprised of the modified chitosan solution with pH adjusted to 9.35 using sodium hydroxide solution.

An emulsion is prepared according to example 1 and then heated to 70° C. before a 2^{nd} modifying compound, SR444, was added to the emulsion at 70° C. The emulsion was then heated to 90° C. in 60 minutes and hold at another 8 hours before cooled down to 25° C. to finish the curing process. The obtained capsule has median particle size of 23.63 micron. The QFO and 1 week leakage of the capsule slurry is 0.35% and 6.19% respectively. The prepared slurry shows no aggregation in heavy duty liquid laundry matrix.

### Example 4: Crosslinked Chitosan Delivery Particles Pre-Modified with CD9055, Acidic Acrylate Oligomer

A modified chitosan solution was prepared by dispersion 42.11 g chitosan powder in 840.00 g water at 70° C. 42.11 g CD9055, an acidic acrylate oligomer was then added to the above chitosan mixture and mixed at 70° C. for 12 hours to obtain CD9055 modified chitosan solution. The pH of the obtained modified chitosan solution is 4.08

A water phase is prepared by weighting 328.00 g CD9055 modified chitosan solution in jacketed reactor at 25° C.

An oil phase is prepared by mixing 112.82 g perfume, 3.08 g Takenate D110 and 28.21 g Isopropyl myristate at 25° C. in a beaker.

The oil phase is added into the water phase under high shear for a period of time to obtain an emulsion at room temperature.

The emulsion is then heated to 40° C. in 30 minutes and then hold at 40° C. for 60 minutes. The emulsion is then heated to 85° C. in 60 minutes and hold for 6 hours to cure the wall. The emulsion is then cooled down to 25° C. in 90 minutes. The obtained capsule has median particle size of 37.74 micron. The QFO and 1 week leakage of the capsule slurry is 0.83% and 41.46% respectively.

### Example 5: Crosslinked Chitosan Delivery Particles Pre-Modified with CD9055, Acidic Acrylate Oligomer and pH Adjustment

A water phase is prepared by weighting 255.78 g CD9055 modified chitosan solution from Example 4 in jacketed reactor at 25° C. The pH of the water phase was then adjusted to 8.23 using 21.5% caustic soda solution. The water phase is still clear after pH adjustment.

An oil phase is prepared by mixing 87.98 g perfume, 2.40 g Takenate D110 and 22.00 g Isopropyl myristate at 25° C. in a beaker.

The oil phase is added into the water phase under high shear for a period of time to obtain an emulsion at room temperature.

The emulsion is then heated to 40° C. in 30 minutes and then hold at 40° C. for 60 minutes. The emulsion is then heated to 85° C. in 60 minutes and hold for 6 hours to cure the wall. The emulsion is then cooled down to 25° C. in 90 minutes. The obtained capsule has median particle size of 25.72 micron. The QFO and 1 week leakage of the capsule slurry is 0.28% and 6.95% respectively. The prepared slurry shows no aggregation in heavy duty liquid laundry matrix.

### Example 6: Crosslinked Chitosan Delivery Particles Pre-Modified with CD9055, Acidic Acrylate Oligomer, and a 2^{nd} Modifying Compound—SR444

A modified chitosan solution is prepared by dispersing 42.11 g chitosan in 840 g water at 70° C. The pH of the above mixture was adjusted to 4.86 using 11.26 g glacial acetic acid. 35.85 g CD9055 was then added to the above chitosan solution and mixed at 70° C. for 12 hours to obtain the CD9055 modified chitosan solution. The pH of the obtained modified chitosan solution is 3.90.

A water phase is prepared by adding 266.70 g CD9055 modified chitosan solution in a jacketed reactor.

An oil phase is prepared by mixing 99.71 g perfume, 2.72 g Takenate D110 and 24.93 g Isopropyl myristate at 25 C in a beaker.

The oil phase is added into the water phase under high shear for a period of time to obtain an emulsion at room temperature.

The obtained emulsion is then heated to 70° C. before a 2^{nd} crosslinker, SR444, was added to the emulsion at 70° C. The emulsion was then heated to 90° C. in 60 minutes and hold at another 8 hours before cooled down to 25° C. to finish the curing process. The obtained capsule has median particle size of 27.52 micron. The QFO and 1 week leakage of the capsule slurry is 0.10% and 34.40% respectively.

### Example 7: Crosslinked Chitosan Delivery Particles Pre-Modified with CD9055, Acidic Acrylate Oligomer and a 2^{nd} Modifying Compound

A modified chitosan solution was prepared by dispersion 42.11 g chitosan powder in 840.00 g water at 70° C. 42.11 g CD9055, an acidic acrylate oligomer was then added to the above chitosan mixture and mixed at 70° C. for 12 hours to obtain CD9055 modified chitosan solution. The pH of the obtained modified chitosan solution is 4.08.

A water phase is prepared by weighting 255.78 g CD9055 modified chitosan solution in jacketed reactor at 25° C.

An oil phase is prepared by mixing 87.98 g perfume, 2.40 g Takenate D110 and 22.00 g Isopropyl myristate at 25° C. in a beaker.

The oil phase is added into the water phase under high shear for a period of time to obtain an emulsion at room temperature. The pH of the emulsion is adjusted to 9.07 using sodium hydroxide solution at 40° C.

The emulsion is then heated to 40° C. in 30 minutes and the pH of the emulsion is adjusted to 9.07 using sodium hydroxide solution. The emulsion is then hold at 40° C. for 60 minutes and then heated to 85° C. in 60 minutes and hold for 6 hours to cure the wall. The emulsion is then cooled down to 25° C. in 90 minutes. The obtained capsule has median particle size of 25.95 micron. The QFO and 1 week leakage of the capsule slurry is 0.66% and 24.91% respectively. The prepared slurry shows no aggregation in heavy duty liquid laundry matrix.

### Example 8: Crosslinked Chitosan Delivery Particles In-Situ Modified with CD9055, Acidic Acrylate Oligomer

A chitosan solution is prepared as in Comparative example 1, but the pH of the chitosan solution is at 5.23.

A water phase is prepared by adding 308.70 g above chitosan solution in a jacketed reactor at 25° C.

An oil phase is prepared by mixing 102.64 g perfume, 2.80 g Takenate D110 and 25.66 g Isopropyl myristate at 25° C. in a beaker.

The oil phase is added into the water phase under high shear for a period of time to obtain an emulsion at room temperature.

The obtained emulsion is then heated to 70° C. before a modifying compound, 10.71 g CD9055 was added to the emulsion at 70° C. The emulsion was then heated to 90° C. in 60 minutes and hold at another 8 hours before cooled down to 25° C. to finish the curing process. The obtained capsule has median particle size of 30.22 micron. The QFO and 1 week leakage of the capsule slurry is 0.49% and 48.08% respectively.

### Example 9: Crosslinked Chitosan Delivery Particles In-Situ Modified with [2-(Acryloyloxy)Ethyl]Trimethylammonium Chloride

A chitosan solution is prepared as in Comparative example 1, but pH of the chitosan solution is at 5.23.

A water phase is prepared by adding 308.70 g above chitosan solution in a jacketed reactor at 25° C.

An oil phase is prepared by mixing 102.64 g perfume, 2.80 g Takenate D110 and 25.66 g Isopropyl myristate at 25° C. in a beaker.

The oil phase is added into the water phase under high shear for a period of time to obtain an emulsion at room temperature.

The obtained emulsion is then heated to 70° C. before a modifying compound, 17.92 g 80% [2-(Acryloyloxy)ethyl]trimethylammonium chloride solution was added to the emulsion at 70° C. The emulsion was then heated to 90° C. in 60 minutes and hold at another 8 hours before cooled down to 25° C. to finish the curing process. The obtained capsule has median particle size of 27.84 micron. The QFO and 1 week leakage of the capsule slurry is 0.29% and 4.62% respectively. The prepared slurry shows no aggregation in heavy duty liquid laundry matrix.

### Example 10: Crosslinked Chitosan Delivery Particles In-Situ Modified with Acrylic Acid

A chitosan stock solution is prepared by dispersing 155.7 g chitosan Chitoclear into 3304 g deionized water while mixing in a jacketed reactor. The pH of the chitosan dispersion is then adjusted to 5.23 using 69.84 g concentrated HCl under agitation. The temperature of the chitosan solution is then increased to 65° C. over 30 minutes, then to 85° C. in 30 minutes, then to 95° C. in 30 minutes, and then held at 95° C. for 2 hours to hydrolyze the chitosan ChitoClear. The temperature is then reduced to 25° C. after the hydrolyzing step over a period of 90 minutes. The pH of the hydrolyzed chitosan solution is 5.31.

A water phase is prepared by mixing 433.6 g of the above chitosan stock solution and in a jacketed reactor at 25° C. An oil phase is prepared by mixing 128.9 g perfume and 32.2 g isopropyl myristate together along with 4.88 g Takenate D-110N at room temperature. The oil phase is added to the water phase under high shear milling to obtain an emulsion with desired particle size. The emulsion is heated to 60° C. in 45 minutes, then to 95° C. in 60 minutes. Once at 95° C., a solution of 2.07 g acrylic acid, 2.07 g RO water, and 5.08 g 21.5% NaOH (prepared in ice bath) added to slurry and then hold at 95° C. for 360 minutes. The temperature is then reduced to 25° C. in 90 minutes. The formed capsules have a median particle size of 30.42 micron.

### Example 11: Crosslinked Chitosan Delivery Particles In-Situ Modified with Acrylic Acid

A crosslinked chitosan capsule slurry is prepared the same as Example 10, except with a solution of 8.29 g acrylic acid, 8.29 g RO water, and 20.31 g 21.5% NaOH (prepared in ice bath) added once at 95° C., instead of a solution of 2.07 g acrylic acid, 2.07 g RO water, and 5.08 g 21.5% NaOH. The formed capsules have a median particle size of 31.25 micron.

### Example 12: Crosslinked Chitosan Delivery Particles In-Situ Modified with 100% Molar Acrylic Acid Add at 25° C.

A crosslinked chitosan capsule slurry is prepared the same as Example 10, except with a solution of 8.29 g acrylic acid, 8.29 g RO water, and 20.31 g 21.5% NaOH (prepared in ice bath) added once an emulsion with desired particle size is attained at 25° C., instead of a solution of 2.07 g acrylic acid, 2.07 g RO water, and 5.08 g 21.5% NaOH added at 95° C. The formed capsules have a median particle size of 31.68 micron. The prepared slurry shows no aggregation in heavy duty liquid laundry matrix.

### Example 13: Crosslinked Chitosan Delivery Particles In-Situ Modified with Acrylic Acid and SR268

A crosslinked chitosan capsule slurry is prepared the same as Example 10, except 1.74 g SR268 is also added right after the solution of 2.07 g acrylic acid, 2.07 g RO water, and 5.08 g 21.5% NaOH added once at 95° C. The formed capsules have a median particle size of 30.83 micron.

### Example 14: Crosslinked Chitosan Delivery Particles In-Situ Modified with 3-Sulfopropyl Acrylate Potassium Salt

A crosslinked chitosan capsule slurry is prepared the same as Example 10, except with adding 6.69 g 3-Sulfopropyl acrylate potassium salt once at 95° C., instead of a solution of 2.07 g acrylic acid, 2.07 g RO water, and 5.08 g 21.5% NaOH. The formed capsules have a median particle size of 30.83 micron. The prepared slurry shows no aggregation in heavy duty liquid laundry matrix.

### Example 15: Crosslinked Chitosan Delivery Particles In-Situ Modified with Glycidyl Trimethylammonium Chloride Add at 25° C.

A crosslinked chitosan capsule slurry is prepared the same as Example 10, except with 10.92 g 80% Glycidyl trimethylammonium chloride added once an emulsion with desired particle size is attained at 25° C., instead of a solution of 2.07 g acrylic acid, 2.07 g RO water, and 5.08 g 21.5% NaOH added at 95° C. The formed capsules have a median particle size of 32.98 micron.

### Example 16: Crosslinked Chitosan Capsule In-Situ Modified with Glycidyl Trimethylammonium Chloride Add at 95° C.

A crosslinked chitosan capsule slurry is prepared the same as Example 10, except with 5.46 g 80% Glycidyl trimethylammonium chloride added once at 95° C., instead of a solution of 2.07 g acrylic acid, 2.07 g RO water, and 5.08 g 21.5% NaOH. The formed capsules have a median particle size of 30.84 micron.

### Example 17: Crosslinked Chitosan Delivery Particles In-Situ Modified with Glycidyl Trimethylammonium Chloride Add at 25° C.

A crosslinked chitosan capsule slurry is prepared the same as Example 10, except with 5.46 g 80% Glycidyl trimethylammonium chloride added once an emulsion with desired particle size is attained at 25° C., instead of a solution of 2.07 g acrylic acid, 2.07 g RO water, and 5.08 g 21.5% NaOH added at 95° C. The formed capsules have a median particle size of 29.61 micron.

### Example 18: Crosslinked Chitosan Delivery Particles In-Situ Modified with Neutralized CD9055, SR268, and KPS

A chitosan stock solution is prepared by dispersing 155.7 g chitosan Chitoclear into 3304 g deionized water while mixing in a jacketed reactor. 1.56 g Potassium Persulfate (KPS) added. The pH of the chitosan dispersion is then adjusted to 5.84 using 57.29 g concentrated HCl under agitation. The temperature of the chitosan solution is then increased to 65° C. over 30 minutes, then to 85° C. in 30 minutes, then to 95° C. in 30 minutes, and then held at 95° C. for 2 hours to hydrolyze the chitosan ChitoClear. The temperature is then reduced to 25° C. after the hydrolyzing step over a period of 90 minutes. The pH of the hydrolyzed chitosan solution is 5.85.

A water phase is prepared by mixing 390.0 g of the above chitosan stock solution and in a jacketed reactor at 25° C. An oil phase is prepared by mixing 115.9 g perfume and 29.0 g isopropyl myristate together along with 4.40 g Takenate D-110N at room temperature. The oil phase is added to the water phase under high shear milling to obtain an emulsion with desired particle size. The emulsion is heated to 60° C. in 45 minutes, then to 95° C. in 60 minutes. Once at 95° C., a solution of 4.52 g CD9055, 4.52 g RO water, and 4.97 g 21.5% NaOH (prepared in ice bath) added to slurry over 1 minute, then 4.20 g SR268 added over 1 minute, then hold at 95° C. for 180 minutes, then added 1.90 g Potassium Persulfate over 1 minute, and then hold at 95° C. for 180 minutes. The temperature is then reduced to 25° C. in 90 minutes. The formed capsules have a median particle size of 28.44 micron. The prepared slurry shows no aggregation in heavy duty liquid laundry matrix.

### Example 19: Crosslinked Chitosan Delivery Particles Modified with Acrylic Acid and KPS

A chitosan stock solution is prepared by dispersing 155.7 g chitosan Chitoclear into 3304 g deionized water while mixing in a jacketed reactor. 1.56 g Potassium Persulfate added. The pH of the chitosan dispersion is then adjusted to 5.84 using 57.24 g concentrated HCl under agitation. The temperature of the chitosan solution is then increased to 65° C. over 30 minutes, then to 85° C. in 30 minutes, then to 95° C. in 30 minutes, and then held at 95° C. for 2 hours to hydrolyze the ChitoClear. The temperature is then reduced to 25° C. after the hydrolyzing step over a period of 90 minutes. The pH of the hydrolyzed chitosan solution is 5.83.

A water phase is prepared by mixing 433.6 g of the above chitosan stock solution and in a jacketed reactor at 25° C. An oil phase is prepared by mixing 129.0 g perfume and 32.0 g isopropyl myristate together along with 4.88 g Takenate D-110N at room temperature. The oil phase is added to the water phase under high shear milling to obtain an emulsion with desired particle size. The emulsion is heated to 60° C. in 45 minutes, then to 95° C. in 60 minutes. 30 minutes after getting to 95° C., a solution of 4.16 g Acrylic Acid, 4.16 g RO water, and 8.04 g 21.5% NaOH (prepared in ice bath) added to slurry over 1 minute, and then hold at 95° C. for 360 minutes. The temperature is then reduced to 25° C. in 90 minutes. The formed capsules have a median particle size of 27.86 micron. The prepared slurry shows no aggregation in heavy duty liquid laundry matrix.

### Example 20: Crosslinked Chitosan Delivery Particles In-Situ Modified with Neutralized CD9055, SR268, and KPS

A chitosan stock solution is prepared by dispersing 155.7 g chitosan Chitoclear into 3304 g deionized water while mixing in a jacketed reactor. 1.56 g Potassium Persulfate added. The pH of the chitosan dispersion is then adjusted to 5.84 using 57.37 g concentrated HCl under agitation. The temperature of the chitosan solution is then increased to 65° C. over 30 minutes, then to 85° C. in 30 minutes, then to 95° C. in 30 minutes, and then held at 95° C. for 2 hours to hydrolyze the ChitoClear. The temperature is then reduced to 25° C. after the hydrolyzing step over a period of 90 minutes. The pH of the hydrolyzed chitosan solution is 5.82.

A water phase is prepared by mixing 432.5 g of the above chitosan stock solution and in a jacketed reactor at 25° C. An oil phase is prepared by mixing 110.0 g perfume and 27.3 g isopropyl myristate together along with 4.15 g Takenate D-110N at room temperature. The oil phase is added to the water phase under high shear milling to obtain an emulsion with desired particle size. The emulsion is heated to 60° C. in 45 minutes, then to 95° C. in 60 minutes. Once at 95° C., a solution of 9.29 g CD9055, 9.29 g RO water, and 10.22 g 21.5% NaOH (prepared in ice bath) added to slurry over 1 minute, then 1.95 g SR268 added over 1 minute, then hold at 95° C. for 180 minutes, then added 1.96 g Potassium Persulfate over 1 minute, and then hold at 95° C. for 180 minutes. The temperature is then reduced to 25° C. in 90 minutes. The formed capsules have a median particle size of 30.26 micron.

### Example 21: Crosslinked Chitosan Delivery Particles Modified with CD9055, SR268, and KPS

A modified chitosan solution was prepared by dispersion 42.11 g chitosan powder in 840.00 g water at 70° C. The pH of the chitosan solution was then adjusted to 4.85 using 11.06 g glacial acetic acid. 35.82 g CD9055, an acidic acrylate oligomer was then added to the above chitosan mixture and mixed at 70° C. for 12 hours to obtain CD9055 modified chitosan solution. The pH of the obtained modified chitosan solution is 3.86.

Weighing 266.7 g the above modified chitosan solution into jacketed reactor at 25° C. and then adjusting the pH of the chitosan solution to 9.27 using 18.84 g 21.5% caustic soda solution at room temperature. The potassium persulfate solution comprising 2.06 g potassium persulfate and 50 g water was then added to the chitosan solution to form a water phase.

An oil phase is prepared by mixing 99.71 g perfume, 2.72 g Takenate D110 and 24.93 g Isopropyl myristate at 25° C. in a beaker.

The oil phase is added into the water phase under high shear for a period of time to obtain an emulsion at room temperature.

The emulsion is heated to 70° C. and then 13 g SR268 was then added to the emulsion. The emulsion is then heated to 90° C. in 60 minutes and held for 8 hours to cure the wall. The emulsion is then cooled down to 25° C. in 90 minutes. The obtained encapsulate, in the form of a water slurry, has median particle size of 43.94 micron. The QFO and 1 week leakage of the capsule slurry is 0.13% and 3.27% respectively. The prepared slurry shows no aggregation in heavy duty liquid laundry matrix.

The water slurries of the core shell particles of the invention, also make possible more concentrated slurries stable in various matrices. The compositions fashioned as a slurry can comprise less than about 25% water, preferably less than about 20% water, more preferably less than about 15% water, even more preferably less than about 12% water, even more preferably less than about 10% water, even more preferably less than about 5% water, by weight of the water relative the weight of the core-shell particles in the composition.

Percent degradation is measured according to the OECD Guidelines for the Testing of Chemicals, test method OECD 301B. A copy is available in www.oecd-ilibrary.org.

The shell of the composition according to the invention has a % degradation of at least 40% degradation after at least 28 days, and of at least 60% degradation after at least 60 days when tested according to test method OECD 301B.

Uses of singular “a,” “an,” are intended to cover both the singular and the plural, unless otherwise indicated herein or clearly contradicted by context. The terms “comprising,” “having,” “including,” and “containing” are to be construed as open-ended terms. All references, including publications, patent applications, and patents, cited herein are hereby incorporated by reference. Any description of certain embodiments as “preferred” embodiments, and other recitation of embodiments, features, or ranges as being preferred, or suggestion that such are preferred, is not deemed to be limiting. The invention is deemed to encompass embodiments that are presently deemed to be less preferred and that may be described herein as such. All methods described herein can be performed in any suitable order unless otherwise indicated herein or otherwise clearly contradicted by context. The use of any and all examples, or exemplary language (e.g., “such as”) provided herein, is intended to illuminate the invention, and does not pose a limitation on the scope of the invention. Any statement herein as to the nature or benefits of the invention or of the preferred embodiments is not intended to be limiting. This invention includes all modifications and equivalents of the subject matter recited herein as permitted by applicable law. Moreover, any combination of the above-described elements in all possible variations thereof is encompassed by the invention unless otherwise indicated herein or otherwise clearly contradicted by context. The description herein of any reference or patent, even if identified as “prior,” is not intended to constitute a concession that such reference or patent is available as prior art against the present invention. No unclaimed language should be deemed to limit the invention in scope. Any statements or suggestions herein that certain features constitute a component of the claimed invention are not intended to be limiting unless reflected in the appended claims.

What is claimed is:

1. A process of forming a population of delivery particles, the delivery particles comprising a core and a shell surrounding the core,
the core comprising a benefit agent,
wherein the shell comprises a reaction product of at least one modified chitosan and at least one electrophile,
the process comprising:
forming a water phase by dissolving or dispersing chitosan in an aqueous acidic medium at a pH of 6.5 or less and a temperature of at least 25° C., the chitosan having free amine moieties;
forming an oil phase comprising combining together at least one benefit agent and at least one electrophile, preferably a polyisocyanate, optionally with an added oil;
forming an emulsion by mixing under high shear agitation the oil phase into an excess of the water phase, thereby forming droplets of the oil phase and benefit agent dispersed in the water phase;
adding to the emulsion or the water phase a water soluble or dispersible modifying compound, the modifying compound comprising one or more of an epoxide, an aldehyde or an α,β-unsaturated compound, the modifying compound reacting with the free amine moieties of the chitosan;
optionally adjusting the pH of the emulsion to a pH of 4 or greater; and
heating the emulsion to at least 40° C., for a time sufficient to form a shell at an interface of the droplets with the water phase, the shell surrounding the core.

2. The process according to claim 1 wherein the delivery particles have a zeta potential of 150 mV or less at pH 4.5.

3. The process according to claim 1 wherein the modifying compound is selected from the group consisting of an epoxide, aldehyde, and an α,β-unsaturated compound, and the modifying compound containing cationic, or anionic groups.

4. The process according to claim 1 wherein the modifying compound is an α,β-unsaturated compound selected from acrylate, alkyl acrylate, α,β-unsaturated ester, acrylic acid, acrylamide, vinyl ketone, vinyl sulfone, vinyl phosphonate, and acrylonitrile.

5. The process according to claim 3 wherein the modifying compound is [2-(acryloyloxy)ethyl]trimethylammonium salt, (3-acrylamidopropyl)trimethylammonium salt, 2-carboxyethyl acrylate, acrylic acid, 2-acrylamido-2-methyl-1-propanesulfonic acid, 2-acrylamido-2-methyl-1-propanesulfonate salt, glycidyl trimethylammonium salt, or combinations thereof.

6. The process according to claim 1 wherein the molar ratio of the modifying compound to the free amine moieties of the chitosan is from 0.1% to 100%, preferably from 10% to 90%, more preferably from 25% to 75%, even more preferably from 25% to 50%.

7. The process according to claim 1 wherein the delivery particles have a modified chitosan content of at least 18 wt %, or even at least 21 wt % based on the weight of the shell.

8. The process according to claim 1 wherein in addition a redox initiator comprising a persulfate or a peroxide is added to the emulsion or the water phase.

9. The process according to claim 1 wherein the electrophile is a polyisocyanate selected from the group consisting of a polyisocyanurate of toluene diisocyanate, a trimethylol propane adduct of toluene diisocyanate, a trimethylol propane adduct of xylylene diisocyanate, 2,4′-methylenediphenyl diisocyanate, [diisocyanato(phenyl)methyl]benzene toluene diisocyanate, tetramethylxylidene diisocyanate, naphthalene-1,5-diisocyanate, 1,4-phenylene diisocyanate, 1,3-diisocyanatobenzene, derivatives thereof, and combinations thereof.

10. A process of forming a population of delivery particles, the delivery particles comprising a core and a shell surrounding the core,
the core comprising a benefit agent and an oil phase,
wherein the shell comprises a reaction product of at least one modified chitosan and at least one electrophile, preferably a polyisocyanate, the modified chitosan comprising a reaction product of chitosan and a modifying compound comprising an epoxide, an aldehyde, or an α,β-unsaturated compound covalently bonded with the chitosan, the process comprising:
dissolving or dispersing chitosan into a water phase, the chitosan having amine moieties,
combining into the water phase at least one of the epoxide, the aldehyde, or the α,β-unsaturated compound, the modifying compound forming a C—N covalent bond with the amine moieties of the chitosan,
optionally adjusting the pH of the water phase to pH 3.0 or higher, and adjusting the temperature of the water phase to 25° C. or greater under mixing for a period of time, to thereby form a modified chitosan, comprising the modifying compound being covalently bonded through C—N bonds with the amine moieties of the chitosan and to maintain the modified chitosan dissolved in the water phase,
providing an oil phase comprising dissolving together at least one benefit agent comprising an oil, and at least one electrophile preferably a polyisocyanate, optionally with a second oil;
forming an emulsion by mixing under high shear agitation the oil phase into the water phase, thereby forming droplets of the oil phase and benefit agent dispersed in the water phase;
heating the emulsion to at least 40° C., for a time sufficient to form the shell at an interface of the droplets with the water phase, the shell surrounding the core.

11. The process according to claim 10 wherein the modifying compound is selected from the group consisting of an epoxide, aldehyde, or an α,β-unsaturated compound, and wherein the modifying compound in addition contains a cationic or an anionic group.

12. The process according to claim 10 wherein the modifying compound is selected from the group consisting of an epoxide, aldehyde, or an α,β-unsaturated compound contains in addition at least one of a cationic, an anionic, or a nonionic group;
wherein the cationic, anionic, or nonionic group is selected from the group consisting of an acidic, a hydroxyl, and a quaternary ammonium group; and,
wherein the α,β-unsaturated compound is selected from the group consisting of acrylate, alkyl acrylate, α,β-unsaturated ester, acrylic acid, acrylamide, vinyl ketone, vinyl sulfone, vinyl phosphonate, and acrylonitrile.

13. The process according to claim 10 wherein the modifying compound is selected from the group consisting of an epoxide, aldehyde, and an α,β-unsaturated compound wherein the modifying compound in addition contains an acidic, hydroxyl, or quaternary ammonium group.

14. The process according to claim 10 wherein the α,β-unsaturated compound is selected from an acrylate, an alkyl acrylate, an α,β-unsaturated ester, an acrylic acid, an acrylamide, a vinyl ketone, a vinyl sulfone, a vinyl phosphonate, or an acrylonitrile.

15. The process according to claim 10 wherein the chitosan is modified with a modifying compound comprising an α,β-unsaturated carbonyl compound.

16. The process according to claim 10 wherein the modifying compound is glycidyl trimethylammonium salt, glycidyl isopropyl ether, glycidyl methacrylate, furfuryl glycidyl ether, glycidol, 1,4-butanediol diglycidyl ether, 2-ethylhexyl glycidyl ether, (3-glycidyloxypropyl) trimethoxysilane, poly(ethylene glycol) diglycidyl ether, trimethylolpropane triglycidyl ether, glutaraldehyde, alginate aldehyde, acrylic acid, acrylate salt, maleic acid, vinyl sulfonic acid, 2-carboxyethyl acrylate, 2-(2-oxo-1-imidazolidinyl)ethyl methacrylate, 2-(2-oxo-1-imidazolidinyl)ethyl methacrylamide, (2-(acryloyloxy)ethyl)trimethylammonium salt, (3-(methacryloylamino)propyl) trimethylammonium salt, N,N-dialkylaminoalkyl acrylate, N,N-dialkylaminoalkyl acrylamide, (3-Acrylamidopropyl)trimethylammonium salt, 3-sulfopropyl acrylate salt, 2-acrylamido-2-methyl-1-propanesulfonic acid and their salts, quaternized vinyl imidazole, diallyl dialkyl ammonium salts, vinyl amine, or combinations thereof.

17. The process according to claim 10 wherein the molar ratio of the modifying compound to free amine moieties of the chitosan is from 0.1% to 99.9%, preferably from 10% to 90%, more preferably from 25% to 75%, even more preferably from 25% to 50%.

18. The process according to claim 10 wherein in addition a redox initiator comprising a persulfate or a peroxide is added to the emulsion or the water phase.

19. The process according to claim 10 wherein the electrophile is a polyisocyanate selected from the group consisting of a polyisocyanurate of toluene diisocyanate, a trimethylol propane adduct of toluene diisocyanate, a trimethylol propane adduct of xylylene diisocyanate, 2,2′-methylenediphenyl diisocyanate, 4,4′-methylenediphenyl diisocyanate, 2,4′-methylenediphenyl diisocyanate, [diisocyanato(phenyl)methyl]benzene toluene diisocyanate, tetramethylxylidene diisocyanate, naphthalene-1,5-diisocyanate, 1,4-phenylene diisocyanate, 1,3-diisocyanatobenzene, derivatives thereof, and combinations thereof.

20. A composition comprising a core-shell microcapsule, the core comprising a benefit agent, the shell comprising a polymer comprising the reaction product of a modified chitosan and an electrophile, preferably a polyisocyanate,
the modified chitosan comprising the reaction product of chitosan and a modifying compound
the core comprising a benefit agent and optionally an oil,
the modifying compound selected from an epoxide, an aldehyde, or an α,β-unsaturated compound containing in addition at least one of a cationic, an anionic, or a nonionic group, wherein the cationic, anionic or nonionic group is selected from an acidic, a hydroxyl, or a quaternary ammonium group, and the α,β-unsaturated compound is selected from acrylic acid, acrylate salt, acrylate, alkyl acrylate, α,β-unsaturated ester, maleic acid, vinyl sulfonic acid, 2-carboxyethyl acrylate, 2-(2-oxo-1-imidazolidinyl)ethyl methacrylate, 2-(2-oxo-1-imidazolidinyl)ethyl methacrylamide, (2-(acryloyloxy)ethyl)trimethylammonium salt, (3-(methacryloylamino)propyl) trimethylammonium salt, N,N-dialkylaminoalkyl acrylate, N,N-dialkylaminoalkyl acrylamide, (3-Acrylamidopropyl)trimethylammonium salt, acrylamide, acrylamide salt, 3-sulfopropyl acrylate salt, 2-acrylamido-2-methyl-1-propanesulfonic acid and their salts, quaternized vinyl imidazole, diallyl dialkyl ammonium salts, vinyl amine, vinyl ketone, vinyl sulfone, vinyl phosphonate, and acrylonitrile;
wherein at least 21 wt % of the shell is comprised of the modified chitosan;
wherein the shell degrades at least 40% when tested according to test method OECD 301B.

21. The composition according to claim 20, the shell comprising 1 to 25 percent by weight of the core-shell microcapsule.

22. The composition according to claim 20 wherein the molar ratio of the modifying compound to free amine moieties of the chitosan is from 0.1% to 99.9%, preferably from 10% to 90%, more preferably from 25% to 75%, even more preferably from 25% to 50%.

23. The composition according to claim 20 wherein the electrophile is selected from the group consisting of a polyisocyanurate of toluene diisocyanate, a trimethylol propane adduct of toluene diisocyanate, a trimethylol propane adduct of xylylene diisocyanate, 2,2′-methylenediphenyl diisocyanate, 4,4′-methylenediphenyl diisocyanate, 2,4′-methylenediphenyl diisocyanate, toluene diisocyanate, tetramethylxylidene diisocyanate, naphthalene-1,5-diisocyanate, 1,4-phenylene diisocyanate, 1,3-diisocyanatobenzene and combinations thereof.

24. The composition according to claim 20 wherein the core-shell microcapsule has a ratio of core to shell up to 99:1, or even 99.5:0.5, on the basis of weight.

25. The composition according to claim 20, comprising a water slurry of a population of core-shell microcapsules, the slurry comprising less than about 25% water, preferably less than about 20% water, more preferably less than about 15% water, even more preferably less than about 12% water, even more preferably less than about 10% water, even more preferably less than about 5% water, by weight of the water relative the weight of the core-shell microcapsules in the composition.

26. The composition according to claim 20 wherein the benefit agent is selected from the group consisting of perfume, fragrance, agricultural active, phase change material, essential oil, lubricant, colorant, preservative, antimicrobial active, antifungal active, herbicide, antiviral active, antiseptic active, antioxidant, biological active, deodorant, emollient, humectant, exfoliant, ultraviolet absorbing agent, corrosion inhibitor, silicone oil, wax, bleach particle, fabric conditioner, malodor reducing agent, dye, optical brightener, antiperspirant active and mixture thereof.

27. The composition according to claim 20 wherein the core-shell delivery particles have a median particle size of from 1 to 200 microns.

28. The composition according to claim 20 wherein the microcapsule is cationic or anionic.

29. The composition according to claim 20 wherein the microcapsule has a zeta potential of 150 mV or less at a pH of 4.5.

30. The composition according to claim 20 wherein the shell degrades at least 60% of its mass after at least 60 days when tested according to test method OECD 301B.
